(19) United States
(12) Patent Application Publication
(10) Pub. No.: US 2015/0023827 A1
(43) Pub. Date: Jan. 22, 2015

(54) Porous Amorphous Alloy Artificial Joint and Manufacturing Method Thereof

(71) Applicant: National Central University, Jhongli City (TW)

(72) Inventors: Shiang Ching JANG, Zhongli City (TW); Chih-Ching HUANG, Kaohsiung City (TW); Jia Bin LI, Zhongli City (TW); Hung Cheng LIN, Zhongli City (TW)

(73) Assignee: National Central University, Jhongli City (TW)

(21) Appl. No.: 14/163,098

(22) Filed: Jan. 24, 2014

(30) Foreign Application Priority Data
Jul. 22, 2013 (TW) 102126068

(51) Int. Cl. A61F 2/30; B22F 3/00; C22C 45/10; B22F 3/14

(52) U.S. Cl. CPC A61F 2/30; B22F 3/14; B22F 3/006; C22C 45/10; A61F 2/3094; B22F 3/1134
USPC 419/2; 148/403

(57) ABSTRACT

The present invention relates to a porous amorphous alloy artificial joint and a manufacturing method thereof The porous amorphous alloy artificial joint is formed of at least one of amorphous alloy compounds represented by Formula 1 to Formula 4 as described in the present specification.

### CROSS REFERENCE TO RELATED APPLICATION

This application claims the benefits of the Taiwan Patent Application Serial Number 102126068, filed on Jul. 22, 2013, the subject matter of which is incorporated herein by reference.

### BACKGROUND OF THE INVENTION

1. Field of the Invention

The present invention relates to a porous amorphous alloy artificial joint having suitable Young's modulus, yield strength, porosity and pore size suitable for cell growth, which is manufactured under various pressures and temperatures by virtue of superplasticity of the amorphous alloy in the supercooled liquid (SCL) region.

2. Description of Related Art

Artificial joint can be considered as one of many important progresses in the medical field in the past few centuries, as it benefits many degenerative arthritis patients. The quality of life for those who have lost mobility can be significantly improved after receiving an artificial hip joint or an artificial knee joint. According to statistics, the number of artificial joint replacement in the United States has reached up to 150,000 or more each year, and gradually increases, indicating that it has become a common orthopedic surgery.

The materials used for making an artificial joint must have good corrosion and impact resistances to prevent peripheral cells from over damage during its use. In addition, biocompatible and porous materials are selected as the material for an artificial joint, in order to facilitate cell growth into the artificial joint to promote recovery of lesion. A typical biomedical porous material is generally made by stainless steel or titanium alloy porous material at a processing temperature of up to 1273 K, resulting in overly high Young's modulus and undesirable stress shielding effect, which easily slows down the recovery rate of the affected region that receives the implant.

Therefore, what is needed is to find an artificial joint suitable for cell growth and having an appropriate Young's modulus and yield strength, in order to improve the current state of the art for artificial joints.

### SUMMARY OF THE INVENTION

An object of the present invention is to provide a porous amorphous alloy artificial joint complying with Young's modulus of the human bones, and having a pore size and porosity suitable for cell growth, to facilitate the application in transplantation of the artificial joints. Another object of the present invention is to provide a method for manufacturing the above porous amorphous alloy artificial joint.

To achieve the above object, the present invention provides a porous amorphous alloy artificial joint formed of at least one of amorphous alloy compounds represented by Formula 1 to Formula 4:

(ZrₐCu_{b}Ni_{c}Al_{d})₁₀₀₋ₓSiₓ,

wherein 45≦a≦75, 15≦b≦45, 5≦c≦15, 5≦d≦10, 1≦x≦10, [Formula 1]

ZrₑCu_{f}Ag_{g}Alₕ)_{100 -y}Si _{y}

wherein 45≦e≦75, 25≦f≦45, 5≦g≦15, 5≦h≦15, 1≦y≦10, [Formula 2]

TiᵢTaⱼSiₖZrₗ,

wherein 30≦i≦80, 0≦j≦20, 1≦k≦20, 5≦1≦40, [Formula 3]

TiₘCuₙZrₒPdₚ,

wherein 40≦m≦75, 30≦n≦40, 5≦o≦15, 10≦p≦20. [Formula 4]

The amorphous alloy compound is preferably at least one selected from the group consisting of Zr₅₃Cu₃₀Ni₉Al₈, (Zr₅₃Cu₃₀Ni₉Al₈)_{100-X}Si_{X}, Zr₄₈Cu₃₆Ag₈Al₈, (Zr₄₈Cu₃₆Ag₈Al₈)_{100-y}Si_{y}, Ti₄₀Zr₁₀Cu₃₆Pd₁₄, Ti₆₀Ta₁₅Si₁₅Zr₁₀, Ti₆₂Ta₁₃Si₁₅Zr₁₀, Ti₆₅Ta₁₀Si₁₅Zr₁₀, Ti₆₀Zr₂₀Ta₅Si₁₅, Ti₆₀Zr₂₂Ta₃Si₁₅, and Ti₄₅Cu₃₅Zr₂₀, wherein 1≦x≦10, 1≦y≦10, more preferably at least one selected from the group consisting of Zr₅₃Cu₃₀Ni₉Al₈ and _{Ti}₄₀Zr₁₀Cu₃₆Pd₁₄, and most preferably Zr₅₃Cu₃₀Ni₉Al₈ and Ti₄₀Zr₁₀Cu₃₆Pd₁₄

The porous amorphous alloy artificial joint has a pore size suitable for cell growth, which is preferably 200-400 and more preferably 250-350 and preferably has a porosity of 40-75%, and more preferably 45-65%. Furthermore, the above-described porous amorphous alloy artificial joint has a Young's modulus and yield strength complying with that of normal joints, wherein the Young's modulus may be 5-25 GPa, and preferably 10-20 GPa, and the yield strength may be 50-350 MPa, and preferably 150-250 MPa.

To prepare the porous amorphous alloy artificial joint, the present invention further provides a method for manufacturing a porous amorphous alloy artificial joint, comprising the following sequential steps:

First, (A) mixing an amorphous alloy power and a water-soluble salt to form a mixture, wherein the porous amorphous alloy power is formed of at least one of amorphous alloy compounds represented by Formula 1 to Formula 4:

(ZrₐCu_{b}Ni_{c}Al_{d})₁₀₀₋ₓSiₓ,

wherein 45≦a≦75, 15≦b≦45, 5≦c≦15, 5≦d≦10, 1≦x≦10, [Formula 1]

ZrₑCu_{f}Ag_{g}Alₕ)_{100 -y}Si _{y}

wherein 45≦e≦75, 25≦f≦45, 5≦g≦15, 5≦h≦15, 1≦y≦10, [Formula 2]

TiᵢTaⱼSiₖZrₗ,

wherein 30≦i≦80, 0≦j≦20, 1≦k≦20, 5≦1≦40, [Formula 3]

TiₘCuₙZrₒPdₚ,

wherein 40≦m≦75, 30≦n≦40, 5≦o≦15, 10≦p≦20. [Formula 4]

Afterward, (B) subjecting the mixture to a hot pressing reaction; and then (C) dissolving the water-soluble salt in the mixture to form the porous amorphous alloy artificial joint.

The step (B) can be performed under an inert gas, such as nitrogen, helium, neon, argon, etc. The hot pressing reaction can be performed at a middle temperature of a supercooled liquid region of the porous amorphous alloy power, preferably ½(Tg+Tx)±20K, and more preferably ½(Tg+Tx)±10K. In the case of Zr₅₃Cu₃₀Ni₉A1₈ and Ti₄₀Zr₁₀Cu₃₆Pd₁₄, the minimum temperature of the hot pressing reaction for Zr₅₃Cu₃₀Ni₉Al₈ is 660K, and the minimum temperature of the hot pressing reaction for Ti₄₀Zr₁₀Cu₃₆Pd₁₄ is 650K.

The hot pressing reaction may be performed under a pressure of 100-500 MPa, and preferably 250-350 MPa. In addition, the reaction time of the hot pressing reaction may be adjusted depending on processing conditions, and is preferably 5-15 minutes, and more preferably 6-12 minutes.

In step (A), the particle size of the amorphous alloy powder may be adjusted as desired, and is preferably 50-300 and more preferably 100-250 In addition, the water-soluble salt can be at least one selected from the group consisting of NaCl, KCl, CaCo₃, and CaF₂, and preferably NaCl.

The amorphous alloy compound is preferably at least one selected from the group consisting of Zr₅₃Cu₃₀Ni₉Al₈, (Zr₅₃Cu₃₀Ni₉Al₈)₁₀₀₋ₓSiₓ, Zr₄₈Cu₃₆Ag₈Al₈, (Zr₄₈Cu₃₆Ag₈Al₈)_{100-y}Si_{y}, Ti₄₀Zr₁₀Cu₃₆Pd₁₄, Ti₆₀Ta₁₅Si₁₅Zr₁₀, Ti₆₂Ta_Si₁₅Zr₁₀, Ti₆₅Ta₁₀Si₁₅Zr₁₀, Ti₆₀Zr₂₀Ta₅Si₁₅, Ti₆₀Zr₂₂Ta₃Si₁₅, and Ti₄₅Cu₃₅Zr₂₀, wherein 1≦x≦10, 1≦y≦10, more preferably at least one selected from the group consisting of Zr₅₃Cu₃₀Ni₉Al₈ and Ti₄₀Zr₁₀Cu₃₆Pd₁₄, and most preferably Zr₅₃Cu₃₀Ni₉Al₈ and Ti₄₀Zr₁₀Cu₃₆Pd₁₄.

In order to manufacture the porous amorphous alloy artificial joint with a preferable pore size, in the step (A), the water-soluble salt is preferably present in an amount of 50-90 vol %, and more preferably 60-70 vol %, based on a total volume of the mixture. Furthermore, a particle size of the water-soluble salt is preferably 150-300 and may be adjusted as desired as well.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows the nucleation curve of Zr₅₃Cu₃₀Ni₉Al₈ according to Examples 1-8 of the present invention.

FIGS. 2A-2H show the sectional views of the porous amorphous alloy artificial joint according to Examples 1-8 of the present invention.

FIGS. 3A-3D show images of the porous amorphous alloy artificial joint of Example 5 at 35, 200, 500, and 150 times magnification, respectively.

FIGS. 4A-4C show images of the porous amorphous alloy artificial joint of Example 8 at 35, 500 and 1000 times magnification, respectively.

FIGS. 5A-5B show images of the porous amorphous alloy artificial joint of Examples 1 and 8, respectively, at 1000 times magnification.

### DETAILED DESCRIPTION OF THE PREFERRED EMBODIMENT

Hereinafter, the actions and the effects of the present invention will be explained in more detail via specific examples of the invention. However, these examples are merely illustrative of the present invention and the scope of the invention should not be construed to be defined thereby. In addition, it is evident that various modifications, structures, processes, and changes may be made thereto without departing from the broader spirit and scope of the present disclosure.

### Examples 1-8

In this Example, Zr-based amorphous alloy material was used. A porous artificial joint suitable for cell growth was prepared under various pressures and temperatures by virtue of the superplasticity of the amorphous alloy in the supercooled liquid (SCL) region. NaCl having different particle sizes was added to the Zr-based amorphous powder having a particle size of 50-300 μm, followed by hot pressing.

[Preparation of Porous Amorphous Alloy Powder]

Zr, Cu, Al, and Ni having a purity of 99.99% were molten into Zr₅₃Cu₃₀Ni₉Al₈ Zr-based alloy ingot by arc-melting (with a power of 350 KW) according to the desired atomic percent of the alloy composition under an argon atmosphere. The alloy ingot was placed in a quartz tube (18 mm in diameter), vacuumed in a quenching melt-spinning chamber to a pressure of 2.0*10⁻² mbar and heated by a high-frequency coil (with a power of 5 KW) under vacuum. After melting (about 1-2 minutes), the molten liquid alloy was ejected onto a water-cooled copper wheel by using argon gas with a pressure of 4-6 kg/cm². The copper wheel was operating at a rotational speed (tangential speed) of 10-20 m/s. For scraping the desired thin strip off from the wheel, a gap was adjusted to less than 1 mm between the copper wheel and the scraper.

The above Zr-based amorphous alloy thin strip was smashed into powder by a blender, and then prepared in the glove box (with an atmosphere of 95% argon, 5% hydrogen). The amorphous alloy powder and tungsten carbide balls were allocated in a weight ratio of 1 (the porous amorphous alloy powder): 10 (tungsten carbide) in a mill jar and ball milled under an atmosphere of pure argon after sealing.

Subsequently, the above substance was placed in a commercial ball mill (SPEX) to perform ball milling, and then the Zr-based amorphous alloy powder with various sizes of (53-297 μm) were sieved out using meshes of different sizes under the protective atmosphere in the glove box.

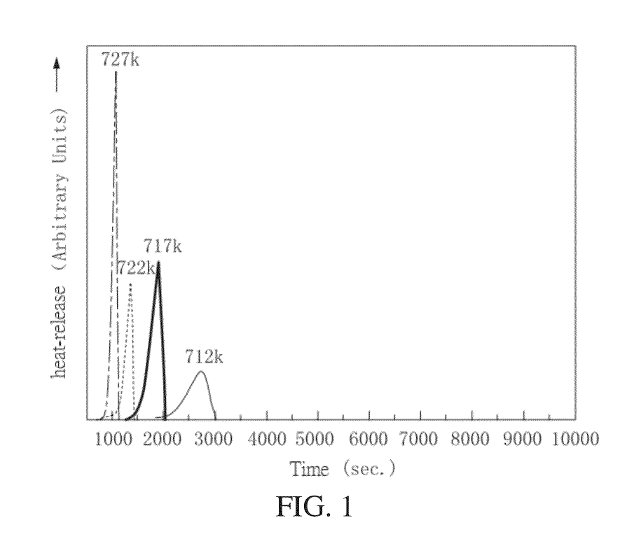

Tg (glass transition temperature), Tx (crystallization temperature) (10-40 K/min) at various rates were analyzed using non-isothermal DSC (Differential Scanning calorimetry), and then the real Tg, Tx was obtained by linear regression. Afterward, an isothermal DSC analysis was performed at the temperature ranging between the real Tg and Tx. The nucleation curve was obtained from the isothermal DSC analysis as shown in FIG. 1. In this Example, the hot pressing reaction was performed at a temperature of 700-740 K and should be completed within 720 seconds (about 12 minutes), or crystallization would otherwise occur.

In addition, as for the Ti₄₀Zr₁₀Cu₃₆Pd₁ Ti-based amorphous alloy powder, the hot pressing reaction was performed at a temperature of 650-680 K for less than 480 seconds.

[Preparation of Zr-Based Porous Amorphous Alloy Artificial Joint]

The above Zr₅₃Cu₃₀Ni₉Al₈ amorphous alloy powder having a density of 6.88 g/cm³ and the NaCl powder having a density of 2.16 g/cm³ were mixed, wherein the particle size of the NaCl powder was between 150-300 μm, and the addition amount of the NaCl powder was calculated according to the following formula:

grams of NaCl=(grams of Zr₅₃Cu₃₀Ni₉Al₈ powder)/(density of Zr₅₃Cu₃₀Ni₉Al₈ powder)*(volume percentage of porous amorphous alloy)*(density of NaCl)

Subsequently, with a given particle size of NaCl (150-300 μm), the hot pressing reaction was performed using the amorphous alloy powders of varying sizes (53-297 μm) under varying hot pressing pressures (100-500 MPa). The reaction conditions are summarized in Table 1:

TABLE 1

| | | Particle size of | volume | |
|---|---|---|---|---|
| | | amorphous alloy | percentage | porous amorphous |
| | MPa | powder (μm) | of NaCl | alloy |
| Example 1 | 300 | 210-297 | 58% | Zr₅₃Cu₃₀Ni₉Al₈ |
| Example 2 | 300 | 149-210 | 60% | Zr₅₃Cu₃₀Ni₉Al₈ |
| Example 3 | 300 | 53-149 | 60% | Zr₅₃Cu₃₀Ni₉Al₈ |
| Example 4 | 300 | 60~ | 60% | Ti₄₀Zr₁₀Cu₃₆Pd₁₄ |
| Example 5 | 300 | 60~ | 60% | Zr₅₃Cu₃₀Ni₉Al₈ |
| Example 6 | 300 | 63-105 | 60% | Zr₅₃Cu₃₀Ni₉Al₈ |
| Example 7 | 500 | 60~ | 60% | Zr₅₃Cu₃₀Ni₉Al₈ |
| Example 8 | 400 | 60~ | 60% | Zr₅₃Cu₃₀Ni₉Al₈ |

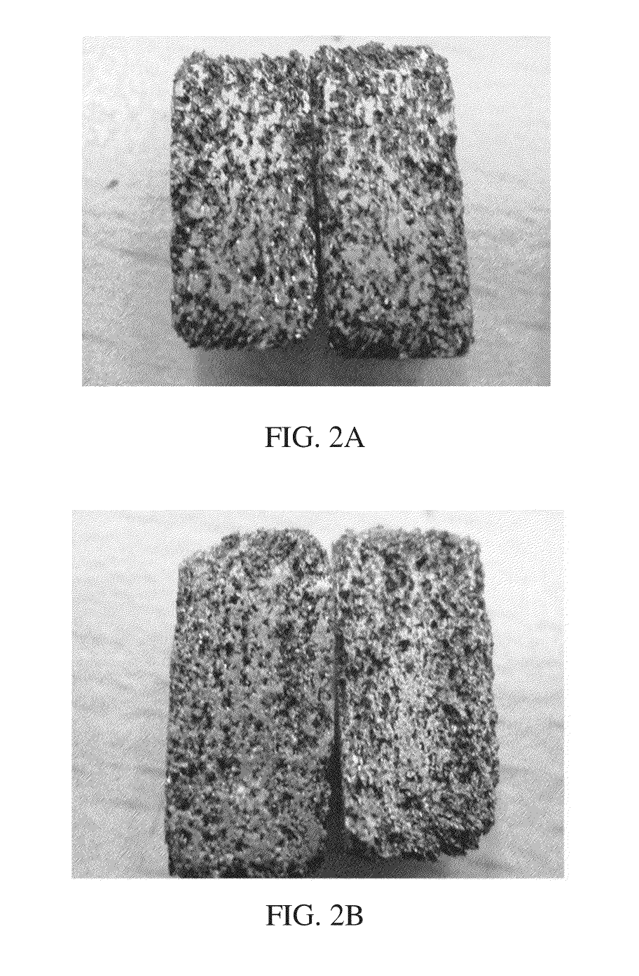

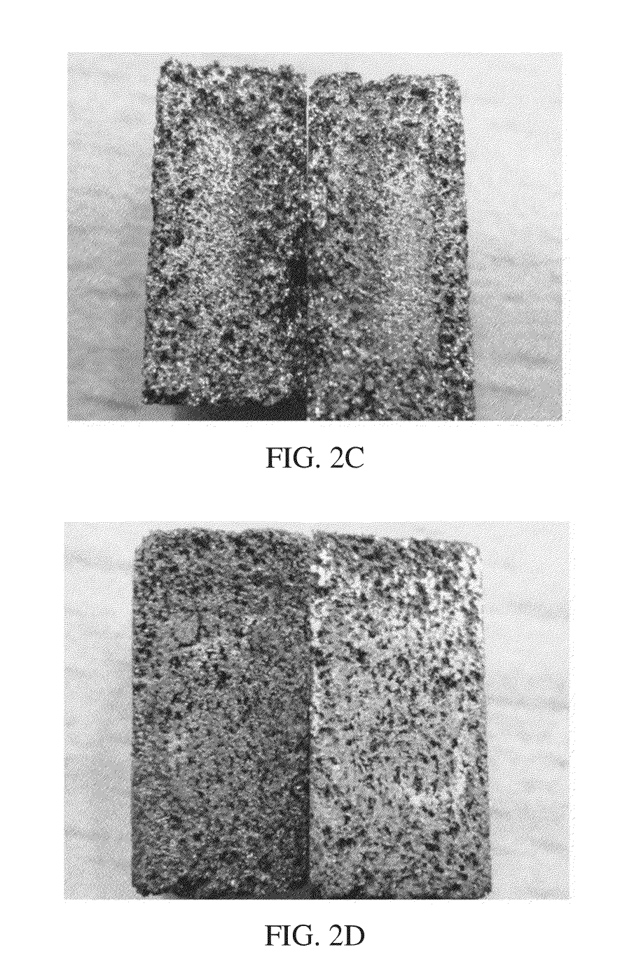

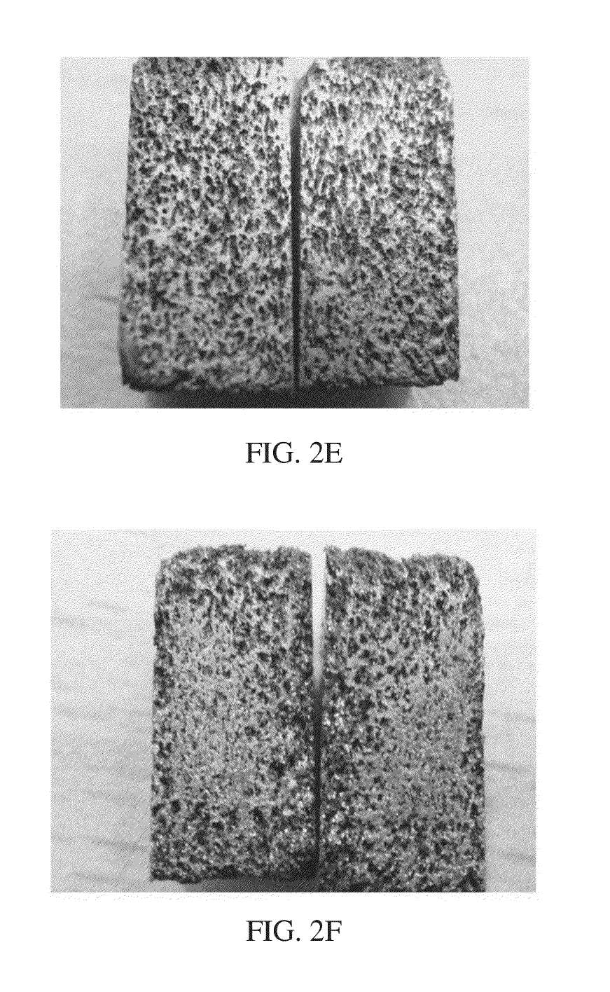

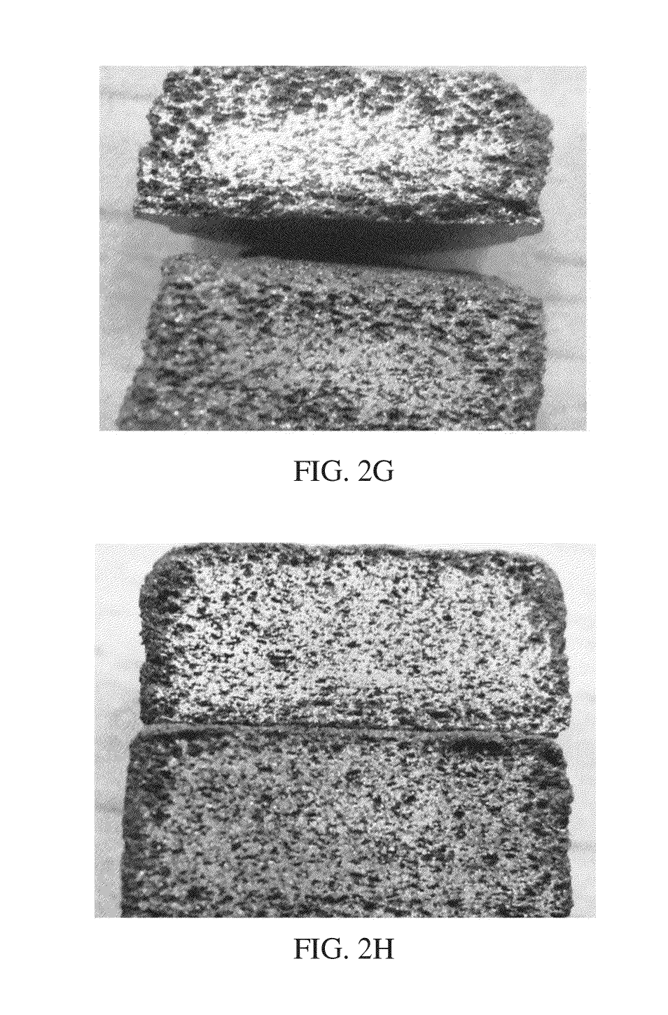

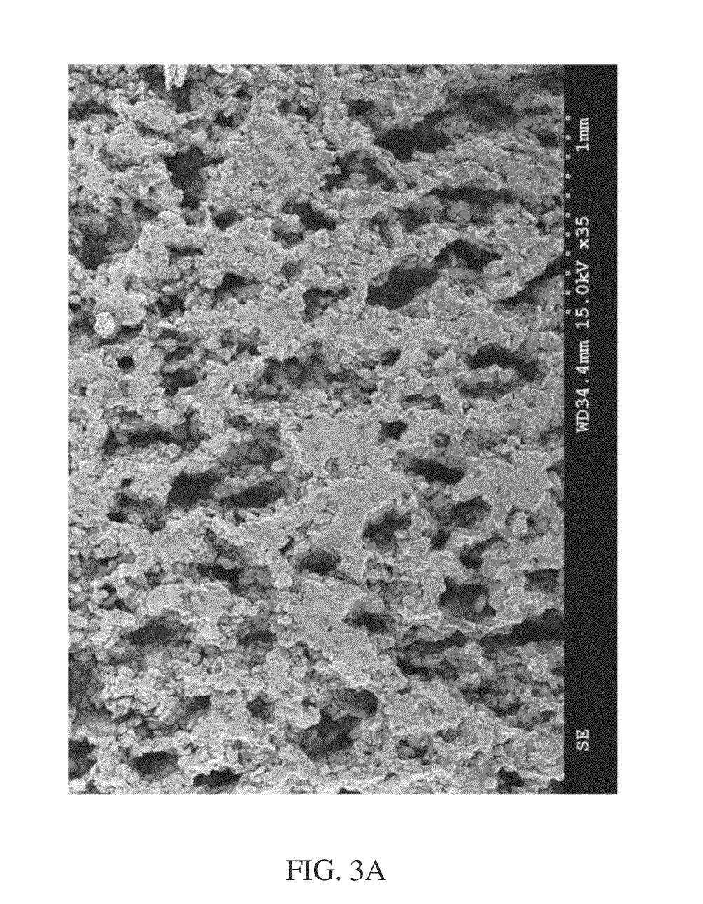

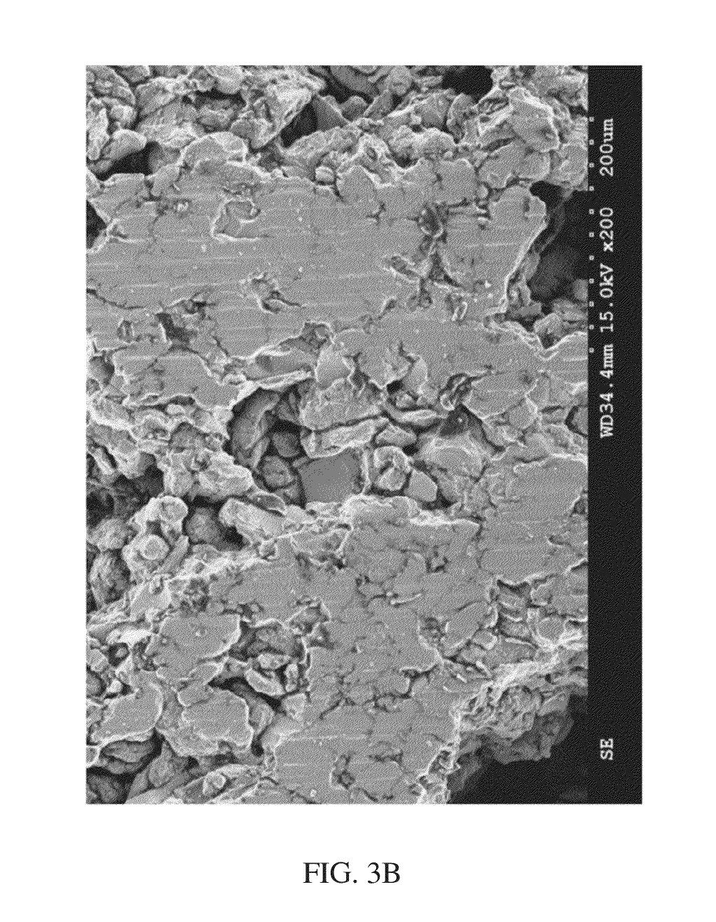

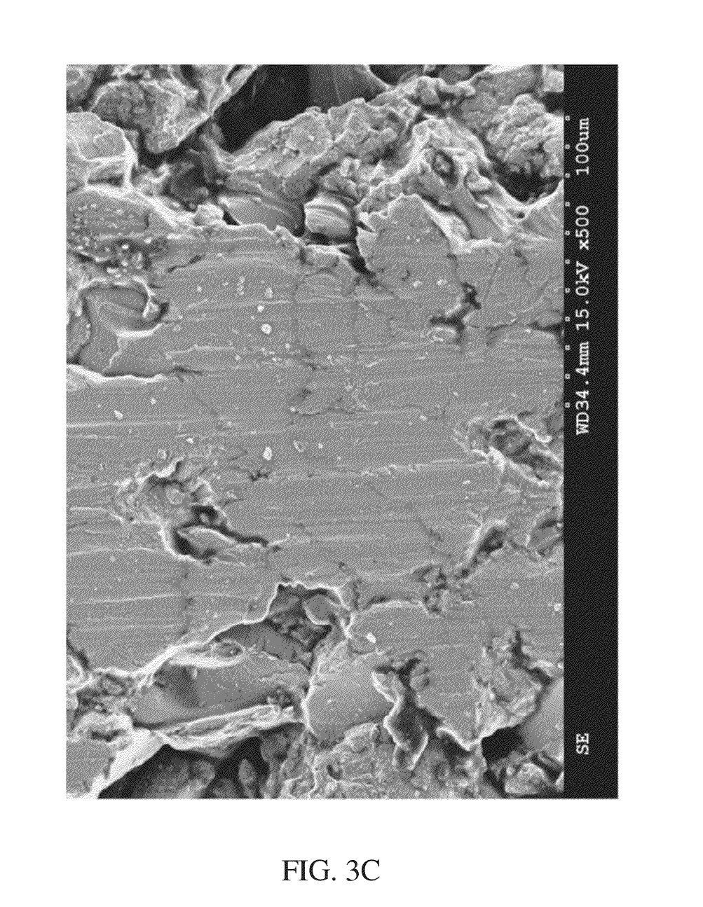

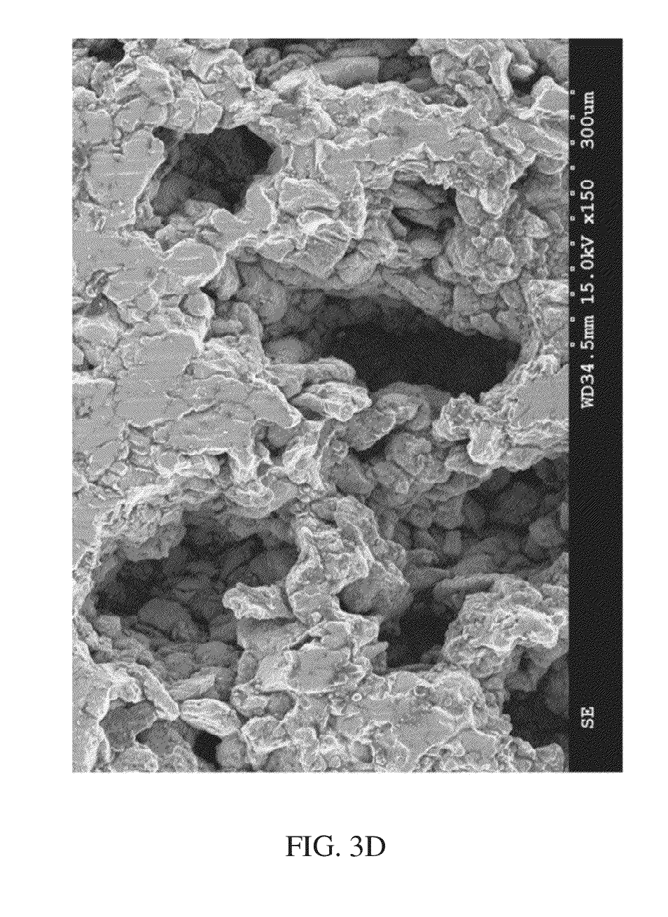

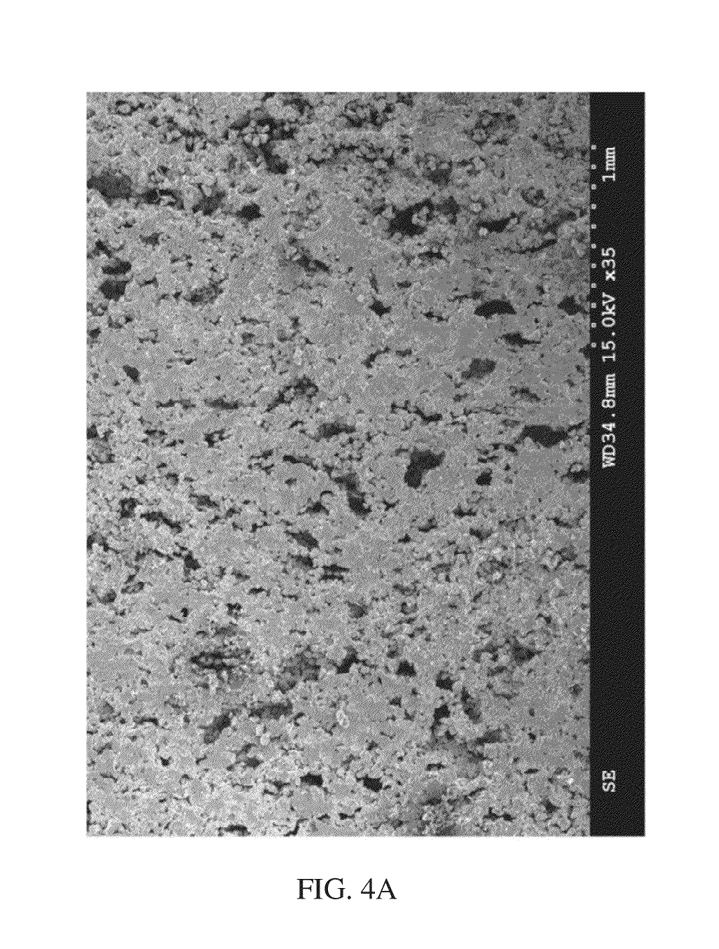

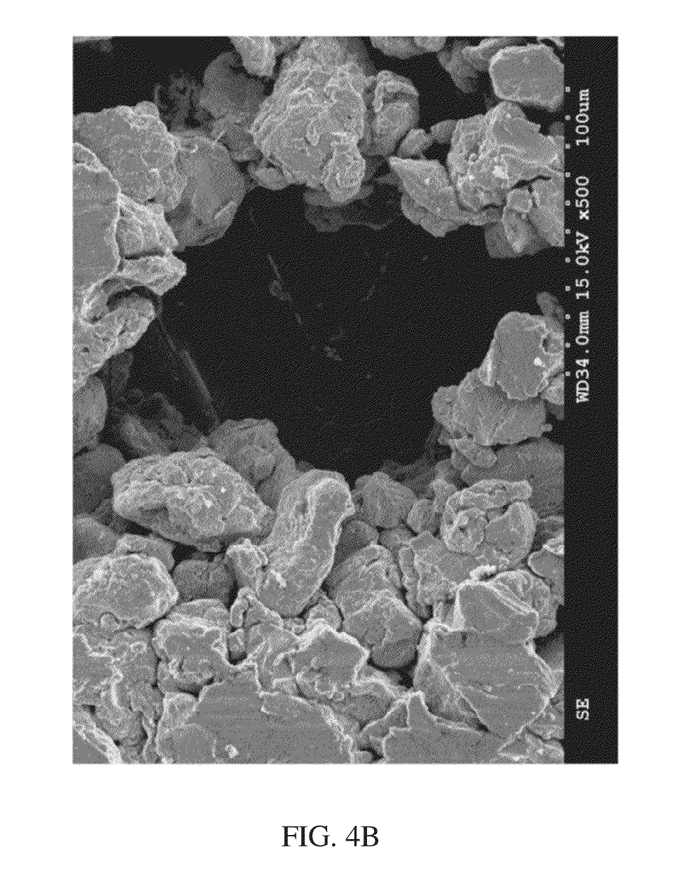

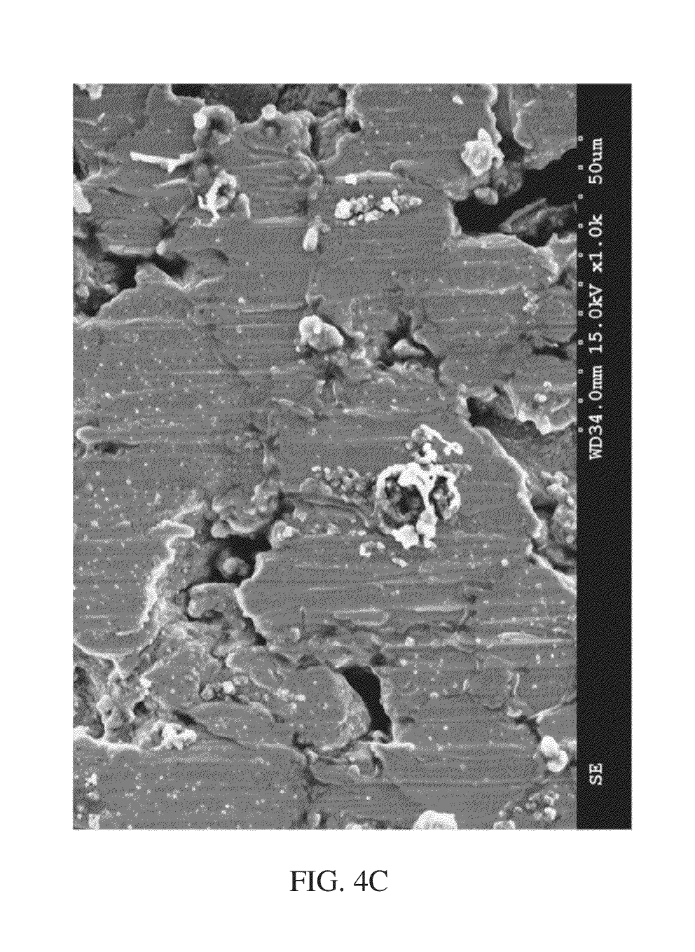

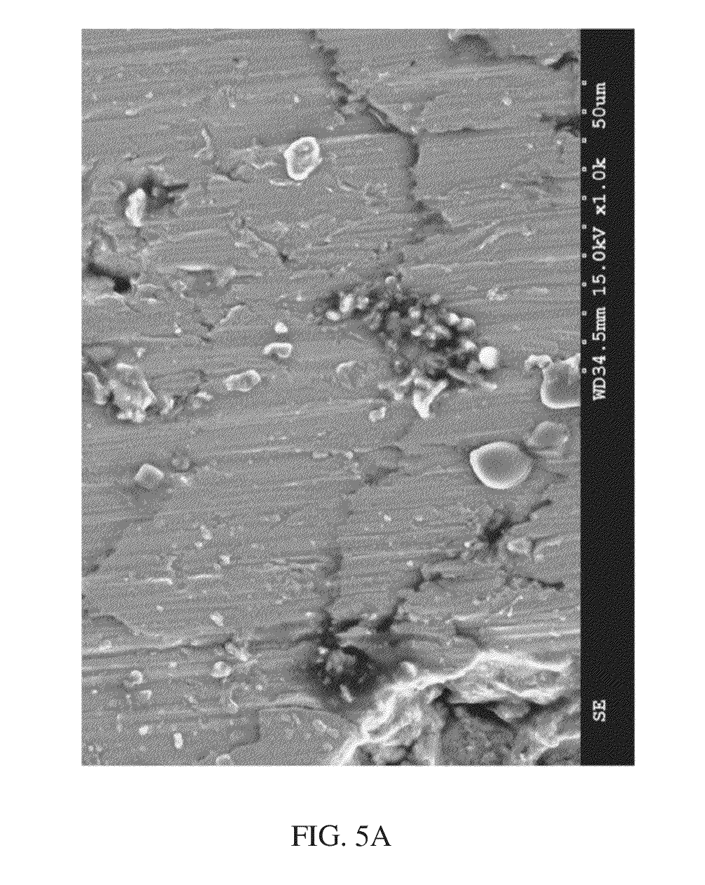

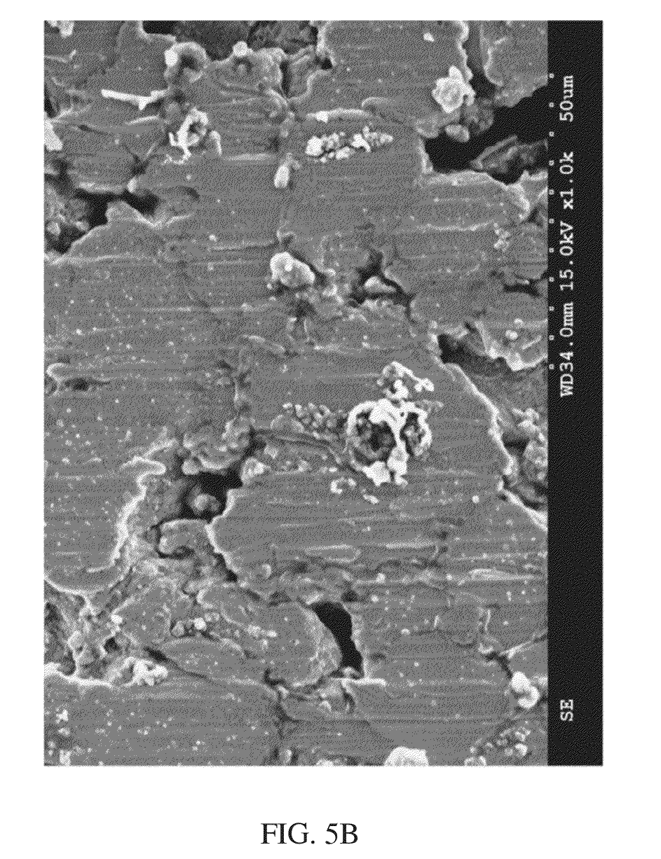

The sectional views of porous amorphous alloy artificial joint in Examples 1 to 8 are shown in FIGS. 2A-2H, wherein the pore size of Examples 1 to 6 were 250±20 μm, the pore size of Example 7 (FIG. 2G) was not measurable due to its non-uniformity, and the pore size of Example 8 was 100±30 μm. The real porosities of Examples 1 to 8 were 40-73%. The Zr-based porous amorphous alloy artificial joint in the most preferable Example 5 had a real porosity of 40-50%, a Young's modulus of 5-25GPa, and a yield strength of 50-320 MPa. Accordingly, the various physical properties of the porous amorphous alloy material can be effectively controlled by choice of the amorphous alloy powders with different particle sizes along with different hot pressing pressures. In the Examples of the present invention, it can be found that under a hot pressing pressure of 300 MPa, the porous artificial joint (with a pore size close to 300 μm) that was most suitable for cell growth could be obtained by mixing the Zr-based amorphous alloy powder with a particle size of 60 μm and 50-90 vol % of NaCl. Among the above, the porous amorphous alloy artificial joint with a porosity of 60% and a pore size of 265±22 μm in Example 5 was most appropriate for cell growth, as shown in FIGS. 3A-3D. The pore size in Example 8 was too small, only 102±30 μm, for cell growth, as shown in FIGS. 4A-4C. Further, referring to FIGS. 5A-5B, no obvious interface was found in the porous artificial joints, indicating a superior metallurgy process has been conducted during the Examples.

Taking Example 5 and 8 as examples, since interstices may be present between the amorphous alloy powders, or NaCl may encapsulate a few amorphous alloy powders during the process, a particle size of larger than 300 μm may be produced by using NaCl of either 150 μm or 300 μm in diameter.

In summary, in the supercooled liquid region (Tg+Tx)/2, under hot pressing pressure of 100-500 MPa, with an amorphous alloy powder having a particle size of 50-300 μm, the porous artificial joint having a high uniformity, meeting the properties of human joint, and suitable for cell growth can be obtained. Compared to the crystalline metal materials which need to be heated to close the melting point to exhibit a near superplastic property, the amorphous alloy powders Zr₅₃Cu₃₀Ni₉Al₈ and Ti₄₀Zr₁₀Cu₃₆Pd₁₄ of the present invention can be thermally shaped at 700-740 K, and 650-680 K, respectively, by hot pressing for an average time of 760-1820 seconds, providing advantages in processing ease and convenience.

It should be understood that these examples are merely illustrative of the present invention and the scope of the invention should not be construed to be defined thereby, and the scope of the present invention will be limited only by the appended claims.

What is claimed is:

1. A porous amorphous alloy artificial joint formed of at least one of amorphous alloy compounds represented by Formula 1 to Formula 4:
(ZrₐCu_{b}Ni_{c}Al_{d})₁₀₀₋ₓSiₓ,
wherein 45≦a≦75, 15≦b≦45, 5≦c≦15, 5≦d≦10, 1≦x≦10, [Formula 1]
ZrₑCu_{f}Ag_{g}Alₕ)_{100 -y}Si _{y}
wherein 45≦e≦75, 25≦f≦45, 5≦g≦15, 5≦h≦15, 1≦y≦10, [Formula 2]
TiᵢTaⱼSiₖZrₗ,
wherein 30≦i≦80, 0≦j≦20, 1≦k≦20, 5≦1≦40, [Formula 3]
TiₘCuₙZrₒPdₚ,
wherein 40≦m≦75, 30≦n≦40, 5≦o≦15, 10≦p≦20. [Formula 4]

2. The porous amorphous alloy artificial joint of claim 1, wherein the amorphous alloy compound is at least one selected from the group consisting of Zr₅₃Cu₃₀Ni₉Al₈, (Zr₅₃Cu₃₀Ni₉Al₈)₁₀₀₋ₓSiₓ, Zr₄₈Cu₃₆Ag₈Al₈, (Zr₄₈Cu₃₆Ag₈Al₈)_{100-y}Si_{y}, Ti₄₀Zr₁₀Cu₃₆Pd₁₄, Ti₆₀Ta₁₅Si₁₅Zr₁₀, Ti₆₂Ta₁₃Si₁₅Zr₁₀, Ti₆₅Ta₁₀Si₁₅Zr₁₀, Ti₆₀Zr₂₀Ta₅Si₁₅, Ti₆₀Zr₂₂Ta₃Si₁₅, and Ti₄₅Cu₃₅Zr₂₀, wherein 1≦x≦10, 1≦y≦10.

3. The porous amorphous alloy artificial joint of claim 1, wherein the amorphous alloy compound is at least one selected from the group consisting of Zr₅₃Cu₃₀Ni₉Al₈ and Ti₄₀Zr₁₀Cu₃₆Pd₁₄

4. The porous amorphous alloy artificial joint of claim 1, wherein a pore size of the porous amorphous alloy artificial joint is 250-350 μm.

5. The porous amorphous alloy artificial joint of claim 1, wherein a porosity of the porous amorphous alloy artificial joint is 45-75%.

6. The porous amorphous alloy artificial joint of claim 1, wherein the porous amorphous alloy artificial joint has a Young's modulus of 5-25 GPa and a yield strength of 50-350 MPa.

7. A method for manufacturing a porous amorphous alloy artificial joint, comprising the following sequential steps:
(A) mixing an amorphous alloy power and a water-soluble salt to form a mixture, wherein the amorphous alloy power is formed of at least one of amorphous alloy compounds represented by Formula 1 to Formula 4:
(ZrₐCu_{b}Ni_{c}Al_{d})₁₀₀₋ₓSiₓ,
wherein 45≦a≦75, 15≦b≦45, 5≦c≦15, 5≦d≦10, 1≦x≦10, [Formula 1]
ZrₑCu_{f}Ag_{g}Alₕ)_{100 -y}Si _{y}
wherein 45≦e≦75, 25≦f≦45, 5≦g≦15, 5≦h≦15, 1≦y≦10, [Formula 2]
TiᵢTaⱼSiₖZrₗ,
wherein 30≦i≦80, 0≦j≦20, 1≦k≦20, 5≦1≦40, [Formula 3]
TiₘCuₙZrₒPdₚ,
wherein 40≦m≦75, 30≦n≦40, 5≦o≦15, 10≦p≦20. [Formula 4]
(B) subjecting the mixture to a hot pressing reaction; and
(C) dissolving the water-soluble salt in the mixture to form the porous amorphous alloy artificial joint.

8. The method for manufacturing a porous amorphous alloy artificial joint of claim 7, wherein, in the step (B), the hot pressing reaction is performed at a middle temperature of a supercooled liquid region of the amorphous alloy power.

9. The method for manufacturing a porous amorphous alloy artificial joint of claim 7, wherein, in the step (B), the hot pressing reaction is performed under an inert gas.

10. The method for manufacturing a porous amorphous alloy artificial joint of claim 7, wherein, in the step (B), the hot pressing reaction is performed at a temperature of ½(Tg+Tx)±20K under an pressure of 100-500 MPa.

11. The method for manufacturing a porous amorphous alloy artificial joint of claim 7, wherein, in the step (B), the hot pressing reaction is performed for 6-12 minutes.

12. The method for manufacturing a porous amorphous alloy artificial joint of claim 7, wherein, in the step (A), the amorphous alloy power has a particle size of 50-300 μm.

13. The method for manufacturing a porous amorphous alloy artificial joint of claim 7, wherein, in the step (A), the water-soluble salt is selected form the group consisting of NaCl, KCl, CaCo₃, and CaF₂.

14. The method for manufacturing a porous amorphous alloy artificial joint of claim 7, wherein, in the step (A), the amorphous alloy power is at least one selected from the group consisting of Zr₅₃Cu₃₀Ni₉Al₈, (Zr₅₃Cu₃₀Ni₉Al₈)₁₀₀₋ₓSiₓ, Zr₄₈Cu₃₆Ag₈Al₈, (Zr₄₈Cu₃₆Ag₈Al₈)_{100-y}Si_{y}, Ti₄₀Zr₁₀Cu₃₆Pd₁₄, Ti₆₀Ta₁₅Si₁₅Zr₁₀, Ti₆₂Ta₁₃Si₁₅Zr₁₀, Ti₆₅Ta₁₀Si₁₅Zr₁₀, Ti₆₀Zr₂₀Ta₅Si₁₅, Ti₆₀Zr₂₂Ta₃Si₁₅, and Ti₄₅Cu₃₅Zr₂₀, wherein 1≦x≦10, 1≦y≦10.

15. The method for manufacturing a porous amorphous alloy artificial joint of claim 7, wherein, in the step (A), the amorphous alloy powder is at least one selected from the group consisting of Zr₅₃Cu₃₀Ni₉Al₈ and Ti₄₀Zr₁₀Cu₃₆Pd₁₄.

16. The method for manufacturing a porous amorphous alloy artificial joint of claim 7, wherein, in the step (A), the water-soluble salt is present in an amount of 50-90 vol % based on a total volume of the mixture.

17. The method for manufacturing a porous amorphous alloy artificial joint of claim 7, wherein, in the step (A), the water-soluble salt has a particle size of 150-300 μm.
